(19) United States
(12) Patent Application Publication
(10) Pub. No.: US 2017/0333595 A1
(43) Pub. Date: Nov. 23, 2017

(54) USE OF POLYMERIC MATERIAL TO REPAIR OSTEOCHONDRAL DEFECTS

(71) Applicant: NEW YORK MEDICAL COLLEGE, Valhalla, NY (US)

(72) Inventors: Paul A. LUCAS, Valhalla, NY (US); Paul D. MARINO, Valhalla, NY (US)

(21) Appl. No.: 15/536,548

(22) PCT Filed: Dec. 16, 2014
(86) PCT No.: PCT/US2014/070533

§ 371 Date: Jun. 15, 2017

(51) Int. Cl. A61L 27/18; A61L 27/38; A61L 27/58

(52) U.S. Cl. CPC A61L 27/18; A61L 27/58; A61L 27/3847; A61L 27/3852; A61L 27/3834; A61L 2300/64; A61L 2430/02; A61L 2430/06

(57) ABSTRACT

Provided is the use of polymeric material, particularly, biodegradable polymeric material in treating osteochondral defects in a subject.

### 1. STATEMENT REGARDING PRIOR DISCLOSURES BY THE INVENTOR OR A JOINT INVENTOR UNDER 37 C.F.R §1.77(b)(6)

Results of studies on the effect of polymeric matrices, specifically PGA discs with and without autologous stem cells on the repair of osteochondral defects was presented by Omowumni Abijola, a medical student working in the laboratory of the inventors, Paul Lucas and Paul Marino on Dec. 18, 2014. A copy of the slides and accompanying paper submitted will be submitted in an Information Disclosure Statement. The authors, Omowumni Abijola, Sherly Abraham and Andrew Grose were not inventors of the subject matter disclosed and claimed in the instant application but were involved working under the supervision of the inventors.

### 2. TECHNICAL FIELD

Provided is the use of polymeric material in treating osteochondral defects in a subject. Said polymeric material could be used to bind and trap autologous stem cells.

### 3. BACKGROUND

Osteochondral defects, such as those encountered in osteoarthritis and joint trauma, are a serious medical problem affecting millions of people in the US. (Buckwalter J A, Mankin H J, Grodzinsky A J. Articular cartilage and osteoarthritis. Instr Course Lect. 2005;54:465-80). Articular cartilage is an avasucular tissue with a low cell turnover rate, which means it has a limited capacity for repair when damaged (Buckwalter J A, Mankin H J, Grodzinsky A J. Articular cartilage and osteoarthritis. Instr Course Lect. 2005;54:465-80; Caplan A I, Elyaderani M, Mochizuki Y, Wakitani S, Goldberg V M. Overview: Principles of cartilage repair and regeneration. Clin Orthop 1997; 342:254-69). There are two types of defects, partial thickness defects and full thickness defects. Partial thickness defects, which do not involve the subchondral bone, are limited to intrinsic repair mechanisms utilizing the limited mitotic and synthetic capabilities of the chondrocytes lining the defect, so repair is insignificant (Caplan A I, Elyaderani M, Mochizuki Y, Wakitani S, Goldberg V M. Overview: Principles of cartilage repair and regeneration. Clin Orthop 1997; 342:254-69; Sokoloff L. Edward A. Dunlop lecture: Cell biology and the repair of articular cartilage. J Rheumatology 1974; 1:1-10). Full thickness defects, which penetrate into the subchondral bone, exhibit extrinsic repair. Extrinsic repair allows undifferentiated mesenchymal elements from the subchondral bone to assist in the repair response, resulting in repair tissue consisting mainly of fibrocartilage (Glimcher M J, Koide S, Shapiro F. Cell origin and differentiation in the repair of full-thickness defects of articular cartilage. J Bone Joint Surg 1993; 75A:532-553). Fibrocartilage does not share the same biochemical properties as articular cartilage (Glimcher M J, Koide S, Shapiro F. Cell origin and differentiation in the repair of full-thickness defects of articular cartilage. J Bone Joint Surg 1993; 75A:532-553; DePalma A F, McKeever C D, Subin D K. Process of repair of articular cartilage demonstrated by histology and autoradiography with tritiated thymidine. Clin Orthop 1966;48:229-242). Thus, the tissue eventually fibrillates and degenerates (Furukawa T, Eyre D, Koide S, Glimcher M J. Biomechanical studies on repair cartilage resurfacing experimental defects in the rabbit knee. J Bone Joint Surg 1980; 62A:79; Coletti J M Jr, Akeson W H, Woo S L. A comparison of the physical behavior of normal articular cartilage and the arthroplasty surface. J Bone Joint Surg 1972; 54A:147; Buckwalter J A, Rosenberg L, Hunziker E B. Articular cartilage: composition, structure, response to injury and methods of facilitating repair. In Ewing, J E, ed. Articular cartilage and knee joint function: Basic science and arthroscopy: New York Raven Press, 1990:19). In both partial and full thickness defects, the body's own natural repair is inadequate and degeneration to osteoarthritis occurs.

Various approaches have been disclosed for treatment of degeneration of articular cartilage. One approach has involved prosthetic joint replacement (arthroplasty) (Mitchell N, Shepard N. The resurfacing of adult rabbit articular cartilage by multiple perforations through the subchondral bone. J Bone Joint Surg 1976; 58A:230-33; Johnson L L. Arthroscopic abrasion arthroplasty: a review. Clin Orthop Relat Res. 2001 October; (391 Suppl):S306-17). However, arthroplasty is not an ideal treatment; it brings with it a risk of infection and the components eventually wear and loosen. Another approach involves biological resurfacing methods such as cartilage-shell, osteochondral implants and joint transplantation (Goldberg V M, H eiple K G. Experimental hemijoint and whole-joint transplantation. Clin Orthop 1983;174:43-53; Gross A E, Silverstein E A, Falk J, Falk R, Langer F. The allotransplantation of partial joints in the treatment of osteoarthritis of the knee. Clin Orthop 1975;108:7-14). However, biological resurfacing is only capable of incomplete regeneration of the joint surface, and the availability of donor materials limits their use (Gross A E, Silverstein E A, Falk J, Falk R, Langer F. The allotransplantation of partial joints in the treatment of osteoarthritis of the knee. Clin Orthop 1975;108:7-14; Sengupta S. The fate of transplants of articular cartilage in the rabbit. J Bone Joint Surg 1974;(B)).

Other approaches have attempted to restore the morphologic and mechanical properties of the joint surface to its original condition using autologous cultured chondrocyte transplantation. However, this method is limited by the availability of donor cartilage and, the difficulty in maintaining the autologous chondrocytes within the defect (Grande D A, Pitman M I, Peterson L, Menche D, Klein M. The repair of experimentally produced defects in rabbit articular cartilage by autologous chondrocyte transplantation. J Orthop Res. 1989;7(2):208-18; Brittberg M, Lindahl A, Nilsson A, Ohlsson C, Isaksson O, Peterson L. Treatment of deep cartilage defects in the knee with autologous chondrocyte transplantation. N Engl J Med. 1994 Oct. 6; 331(14):889-95; Brittberg M, Nilsson A, Lindahl A, Ohlsson C, Peterson L. Rabbit articular cartilage defects treated with autologous cultured chondrocytes. Clin Orthop 1996;326:270-83). Also, several reports also indicate that the repair degenerates with time (Richardson J B, Caterson B, Evans E H, Ashton B A, Roberts S. Repair of human articular cartilage after implantation of autologous chondrocytes. J Bone Joint Surg 1999;818:1064-68; Breinan H A, Martin S D, Hsu H P, Spector M. Healing of canine articular cartilage defects treated with microfracture, a type-II collagen matrix, or cultured autologous chondrocytes. J Orthop Res 2000;18:781-89; Lee C R, Grodzinsky A J, Hsu H P, Martin S D, Spector M. Effects of harvest and selected cartilage repair procedures on the physical and biochemical properties of articular cartilage in the canine knee. J Orthop Res 2000;18:790-99). One approach to address this issue has involved using various delivery vehicles/substrates for promoting the growth of such cells (see, for example US Patent Appln. Nos. 20130004541 (PLA/PGA nanofiber incorporating a heparin/fibrin delivery system), WO2008050185 (cell coated substrate, particularly a PLA/PGA lattice), US Patent Appln. Pub. No. 200710043782 (tissue engineering cartilage prepared by coating a matrix with autologous marrow matrix stem cells and culturing in the presence of TGF-betal, IGF-1 and insulin), US Patent Appln. Pub. No. 20130236971 (hydrogel scaffolds), WO2013073941(dextran based polymer comprising platelet rich plasma lysate), US Patent Appln. Pub. No. 20130197662 (a collagen based material).

Another approach has involved using mesenchymal stem cells derived from a variety of sources, including but not limited to, bone marrow (Wakitani S, Goto T, Pineda S J, Young R G, Mansour J M, Caplan A I, Goldberg V M. Mesenchymal cell-based repair of large, full-thickness defects of articular cartilage. J Bone Joint Surg 1994;76A:579-92; Im G I, Kim D Y, Shin J H, Hyun C W, Cho W H. Repair of Cartilage defect in the rabbit with cultured mesenchymal stem cells from bone marrow. J Bone Joint Surg 2001; 83B:289-94; Angele P, Kujat R, Nerlich M, Yoo J, Goldberg V, Johnstone B. Engineering of osteochondral tissue with bone marrow mesenchymal progenitor cells in a derivatized hyaluronan-gelatin composite sponge. Tissue Engineering 1999;5:545-54; Diduch D R, Jordan L C, Mieisch C M, Balian G. Marrow stromal cells embedded in alginate for repair of osteochondral defects. Arthroscopy 2000;16:571-77), adipose (Merceron C, Vinatier C, Clouet J, Colliec-Jouault S, Weiss P, Guicheux J. Adipose-derived mesenchymal stem cells and biomaterials for cartilage tissue engineering. Joint Bone Spine. 2008 December; 75(6):672-4), embryoid bodies (US Patent Appln. Pub. No. 20130230601), costal cartilage (US Patent Appln. Pub. No. 20090228105), synovium (US Patent Appln. Pub. Nos. 20100178274 or US20130195810). Pluripotent stem cells have also been used in another approach (see, for example, WO2013013105, US Patent Pub. No. 20110177043). However, all of these are relatively ineffective unless first induced to a chondrogenic lineage in vitro (Perka C, Schultz O, Spitzer R S, Lindenhayn K. The influence of transforming growth factor betal on mesenchymal cell repair of full-thickness cartilage defects. J Biomed Mater Res 2000; 52:543-52; Zscharnack M, Hepp P, Richter R, Aigner T, Schulz R, Somerson J, Josten C, Bader A, Marquass B. Repair of chronic osteochondral defects using predifferentiated mesenchymal stem cells in an ovine model. Am J Sports Med. 2010 September; 38(9):1857-69). While this improved the repair of the cartilage, prior induction of the cells to chondrocytes results in suboptimal repair of the subchondral bone.

The current standard treatment for osteochondral defects is microfracture (see, for example, http://orthoinfo.aaos.org/topic.cfm?topic=a00422, http://www.nlm.nih.gov/medlineplus/ency/article/007255.htm, Falah M, Nierenberg G, Soudry M, Hayden M, Volpin G. Treatment of articular cartilage lesions of the knee. Int Orthop. 2010 Jun;34(5):621-30.) In this procedure, small holes are punched through the bone at the base of the defect into the underlying trabecular bone and its marrow spaces. Marrow wells up into the defect, and this marrow contains autologous stem cells which participate in the repair of the defect. The repair tissue is fibrocartilage which degrades over time (Steadman J R, Rodkey W G, Singleton S B, Briggs K K. Microfracture technique for full-thickness chondral defects: technique and clinical results. Operat Tech Orthop 7:300-307, 1997, Kreuz P C, Erggelet C, Steinwachs M R, Krause S J, Lahm A, Niemeyer P, Ghanem N, Sudkamp N. Is microfracture of chondral defects in the knee associated with different results in patients aged 40 years or younger? Arthroscopy 22(11):1180-1186, 2006.). However, microfracture does provide relief from pain. Any innovation that improves the result of microfracture would benefit patients.

Multipotent adult stem cells (MASCs) (also referred to as in previous papers as “mesenchymal stem cells and in a patent as “pluripotent adult stem cells) have also been isolated from skeletal muscle and have been found to be capable of differentiating into tissues from all three dermal layers, (U.S. Pat. No. 7,259,011; Pate, D W, Southerland S S, Grande, D. A., Young, H. E., and Lucas, P. A. Isolation and differentiation of mesenchymal stem cells from rabbit muscle. Surgical Forum 44: 587-589, 1993; Lucas, P. A., Calcutt, A. F., Southerland, S. S., Wilson, A., Harvey, R. Warejcka, D., and Young, H. E. A population of cells resident within embryonic and newborn rat skeletal muscle is capable of differentiating into multiple mesodermal phenotypes. Wound Repair and Regeneration, 3: 449-460, 1995; Young H E, Steele T A, Bray R A, Detmer K, Blake L W, Lucas P A, Black A C Jr. Human pluripotent and progenitor cells display cell surface cluster differentiation markers CD10, CD13, CD56, and MHC class-I. Proc Soc Exp Biol Med. 221(1):63-71, 1999; Young H E, Steele T A, Bray R A, Hudson J, Floyd J A, Hawkins K, Thomas K, Austin T, Edwards C, Cuzzourt J, Duenzl M, Lucas P A, Black A C Jr. Human reserve pluripotent mesenchymal stem cells are present in the connective tissues of skeletal muscle and dermis derived from fetal, adult, and geriatric donors. Anat Rec. 2001 Sep. 1; 264(1):51-62; Schultz S S, Lucas P A. Human stem cells isolated from adult skeletal muscle differentiate into neural phenotypes. J Neurosci Methods. 2006 Apr. 15; 152(1-2):144-55; Schultz S S, Abraham S, Lucas P A. Stem cells isolated from adult rat muscle differentiate across all three dermal lineages. Wound Repair Regen. 2006 March-April; 14(2):224-31). MASCs grown into a polyglycolic (PGA) felt matrix for 2 weeks and implanted in a critical sized osteochondral defect in the femoropatellar groove of adult rabbits showed histological signs of regeneration at 12 weeks post-op (Grande D A, Southerland S S, Manji R, Pate D W, Schwartz R E, Lucas P A. Repair of articular cartilage defects using mesenchymal stem cells. Tissue Engineering 1995;1:345-53). The implantation of the PGA felt matrix did not result in any detectable regeneration (see, for example Taub, P J, Jervis, Y, Spangler, M., Mason, J M, and Lucas, P A. Bioengineering of calvaria with adult stem cells. Plastic Reconstructive Surgery 2009,123(4): 1178-85; Grande, D. A., Southerland, S. S., Manji, R., Pate, D. W., Schwartz, R. E., and Lucas, P. A. Repair of articular cartilage defects using mesenchymal stem cells. Tissue Engineering 1995, 1: 345-353,).

### 4. SUMMARY

Provided is a method for growing new cartilage, including but not limited to articular cartilage and optionally subchondral bone in a subject in need thereof comprising administering to a site where articular cartilage and optionally subchondral bone is needed an amount of a polymeric material suitable for proliferation and differentiation of autologous stem cells into articular cartilage or articular cartilage and subchondral bone, effective for growing said articular cartilage and said subchondral bone. As noted above, said polymeric material could be used to bind and trap autologous stem cells. In a particular embodiment, said polymeric material is treated with a preparation comprising uncultured autologous stem cells for a time sufficient for said autologous stem cells to attach to said polymer.

The method may further comprise a microfracture procedure to articular cartilage and/or subchondral bone in said subject immediately prior to administration and particularly, implantation of said polymeric material.

In a particular embodiment, said polymeric material is biodegradable. In a specific embodiment, said polymeric material may include polyglycolic acid.

In another particular embodiment, said subject has a defect in both the cartilage and bone. In yet another particular embodiment, said subject is afflicted with an osteochondral defect.

### 5. BRIEF DESCRIPTION OF FIGURES

FIG. 1 shows A. Empty Defect and B. Defect treated with PGA with respect to the femoropatellar groove 26 weeks post implantation. A line represents 1 mm.

FIG. 2 is a higher power picture of the defects in the femoropatellar groove at 26 weeks. A. Empty Defect and B. Defect treated with PGA with respect to the femoropatellar groove 26 weeks post implantation. A line represents 100 μm.

FIG. 3 shows A. Empty Defect and B. Defect treated with PGA with respect to the femoropatellar groove 52 weeks post implantation. A line represents 1 mm.

FIG. 4 is a higher power picture of defects in the femoropatellar groove at 52 weeks. A. Empty Defect and B. Defect treated with PGA with respect to the femoropatellar groove 52 weeks post implantation. Line represent 100 μm.

FIG. 5 shows A. Empty Defect and B. Defect treated with PGA with respect to the medial condyle 26 weeks post implantation. Line represents 1 mm.

FIG. 6 is a higher power picture of the defects in the medial condyle at 26 week A. Empty Defect and B. Defect treated with PGA with respect to the medial condyle 26 weeks post implantation. Line represents 100 μm.

FIG. 7 shows A. Empty Defect and B. Defect treated with PGA with respect to the medial condyle 52 weeks post implantation Line represents 1 mm.

FIG. 8 is a higher power picture of the defects in the medial condyle at 26 week A. Empty Defect and B. Defect treated with PGA with respect to the medial condyle 26 weeks post implantation Line represents 100 μm.

### 6. DETAILED DESCRIPTION OF SPECIFIC EMBODIMENTS

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

In the description and claims of the present application, each of the verbs, “comprise,” “include” and “have,” and conjugates thereof, are used to indicate that the object or objects of the verb are not necessarily a complete listing of components, elements or parts of the subject or subjects of the verb.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended.

The term “defect” as used herein refers to an imperfection that impairs worth or utility or the absence of something necessary for completeness or perfection; or a deficiency in function. The term defect as used herein is not limited to acquired defects, for example defects from damage from, for example, diseases such as osteoarthritis or arthritis, injury or wear, but the term defects also encompasses defects due to non-acquired or existing defects, for example congenital or developmental defects.

As defined herein, an “osteochondral defect” is a focal area of articular damage with cartilage damage and injury of the adjacent subchondral bone and may be due to Osteochondritis dissecans (OCD), avascular necrosis, or trauma. In a particular embodiment said osteochondral defect is due to osteoarthritis or alternatively, joint trauma.

As used herein, the term “articular cartilage”, is understood to mean any cartilage tissue, that biochemically and morphologically resembles the cartilage normally found on the articulating surfaces of mammalian joints.

As used herein, the term “polymer” in the present application is intended to mean without limitation a polymer solution, polymer suspension, a polymer particulate or powder and a polymer micellar suspension.

As used herein, the term “bioresorbable” refers to the ability of a material to be reabsorbed in vivo. The absorbable polymer material can is selected from the group consisting of polyglycolic acid (PGA), polylactic acid (PLA), poly(lactic-co-glycolic) acid (PLGA), polyanhydride, polycapralactone (PCL), polydioxanone and polyorthoester. The bioabsorbable polymer material also can be composite material that comprises an absorbable polymer material and other materials.

As used herein, the term “biodegradable” as used herein denotes a composition that is not biologically harmful and can be chemically degraded or decomposed by natural effectors (e.g., weather, soil bacteria, plants, animals).

As used herein, the term “glycolide” is understood to include polyglycolic acid. Further, the term “lactide” is understood to include L-lactide, D-lactide, blends thereof, and lactic acid polymers and copolymers.

The term “polyglycolic acid”, “poly(glycolic) acid and “PGA” are used interchangeably herein, refer to a polymer of glycolic acid. The term “polylactic acid”, “poly(lactic) acid” and “PLA” are used interchangeably herein, refer to a member of the polyester family, in particular the poly(α-hydroxyl acid) family, and refers to a polymer of lactic acid molecules. The terms PLA and polylactic acid are intended to encompass all isometric forms of poly(lactic)acid, for example d( ) 1(+) and racemic (d,l) and the polymers are usually abbreviated to indicate the chirality. Poly(I)LA and poly(d)LA are semi-crystalline solids.

As used herein, the term “porous” as used herein, refers to small indentations or void spaces on the surface of the substrate in which cells and other materials can adhere to. For the purposes of the present application, the void spaces are on the surface and substantially located near the surface of the substrate.

### Polymeric Material

In a particular embodiment, the polymeric material is porous material, including but not limited to polymeric mesh or sponge. The polymeric material can in a specific embodiment, may be in the form of felt. In another particular embodiment, the polymeric material is biodegradable over a time period of between about two weeks to about two years. The polymeric material can be manufactured or constructed using commercially available materials. This material is typically derived from a natural or a synthetic polymer. Biodegradable polymers are preferred, so that the newly formed cartilage can maintain itself and function normally under the load-bearing present at synovial joints. Synthetic polymers are preferred because their degradation rate can be more accurately determined and they have more lot to lot consistency and less immunogenicity than natural polymers. Natural polymers that can be used include but are not limited to proteins such as collagen, albumin, and fibrin; and polysaccharides such as alginate and polymers of hyaluronic acid. Synthetic polymers include both biodegradable and non-biodegradable polymers. Examples of biodegradable polymers include but are not limited to polymers of hydroxyl acids such as polylactic acid (PLA), polyglycolic acid (PGA), and polylactic acid-glycolic acid (PLGA), polyorthoesters, polyanhydrides, polyphosphazenes, polydioxanone, those described in WO2007022149 and US20070036842, GELFOAM® polycaprone. Polymeric materials used in the instant method may be obtained from commercial sources such as Biomedical Structures, Inc., Ethicon, or Pfizer and combinations thereof. Non-biodegradable polymers include polyacrylates, polymethacrylates, ethylene vinyl acetate, and polyvinyl alcohols. These should be avoided since their presence in the cartilage will inevitably lead to mechanical damage and breakdown of the cartilage.

The starting material may, in a specific embodiment, include a bioabsorbable polymer, such as polyglycolic acid, or poly-L lactide, or copolymers that include one of each. The polymer composition, as well as method of manufacture, can be used to determine the rate of degradation. For example, mixing increasing amounts of polyglycolic acid with polylactic acid decreases the degradation time. Examples of such polymeric materials include but are not limited to

The polymeric material is preferably shaped to be slightly larger than the defect. In most cases, this can be achieved by trimming the polymer fibers with scissors or a knife. Alternatively, the polymer can be cast from a polymer solution formed by heating or dissolution in a volatile solvent.

In a particular embodiment, said polymeric material is treated with a preparation comprising uncultured autologous stem cells for a time sufficient for said autologous stem cells to become attached to said polymer. In a specific embodiment, said polymer is incubated with said preparation for about 4 to about 6 hours. Said autologus cells in said preparation may be derived from various tissues including but not limited to bone marrow or adipose tissue using procedures known in the art (see, for example, Sampson S, Botto-van Bemden A, Aufiero D, Autologous bone marrow concentrate: review and application of a novel intra-articular orthobiologic for cartilage disease Phys Sportsmed. 2013 September; 41(3):7-18 and Peeters C M, Leijs M J, Reijman M, van Osch G J, Bos P K. Safety of intra-articular cell-therapy with culture-expanded stem cells in humans: a systematic literature review. Osteoarthritis Cartilage. 2013 October; 21(10):1465-73).

### Conditions to be Treated

The polymeric material can be used to create or supplement connective tissue as required. In some cases, this will be to repair existing defects, particularly osteochondral defects. In a particular embodiment, said defect may be a cartilage defect that covers or adversely affects a surface area larger than about 1 square centimeter (cm), for example, at least about 1 cm², or at least about 1.5 cm² or at least about 2 cm², or at least about 3 cm² or greater than about 3 cm² or any surface area size or diameter in between about 1 cm² and about 10 cm². In another particular embodiment said method may be used treatment of traumatic fractures, pathologic fractures, stress fractures, congenital defects or fractures, or defects resulting from diseases such as osteoarthritis or arthritis.

Said polymeric material may be administered using procedures known in the art. In a particularly embodiment, as noted above, the polymeric material is preferably slightly larger than the defect and implanted into the site of the defect either by an open procedure or arthroscopically. If the defect is very large, the matrix may be anchored in the defect by 1-6 sutures in the periphery.

In a particular embodiment, articular cartilage and/or subchondrondral bond is subject to microfracture immediately prior to implantation of said polymeric material using procedures known in the art (see, for example. http://orthoinfo.aaos.org/topic.cfm?topic=a00422, http://www.nlm.nih.gov/medlineplus/ency/article/007255.htm, Falah M, Nierenberg G, Soudry M, Hayden M, Volpin G. Treatment of articular cartilage lesions of the knee. Int Orthop. 2010 June; 34(5):621-30.).

### 6. EXAMPLE

### Materials and Methods

Seeding MASCs into PGA Felt

Polyglycolic acid (PGA) mesh, composed of non-woven fiber mats with 12 to 14 μm diameter fibers at a density of 55 to 65 mg/cm³ and 2 mm thick (obtained in a 20×30 cm sheet, Concordia Manufacturing, Coventry R.I.) was cut into circles 10 mm in diameter and sterilized with alcohol. Immediately prior to implantation, a 4 mm trephine was used to remove a 4 mm diameter plug from the PGA felt disc to be press-fitted into the defect. Experience has shown that the implant will remain in the defect without further manipulation and this seemed to be confirmed in this study. The PGA discs were washed 3× with PBS prior to implantation.

### Osteochondral Defect Formation and Experimental Groups

The use of animals was in compliance with the recommendation of the Panel on Euthanasia of the American Veterinary Medicine Association and was performed under approval by the IACUC committee at New York Medical College. Defects were 3 mm in diameter and were prepared as described previously (Grande D A, Southerland S S, Manji R, Pate D W, Schwartz R E, Lucas P A. Repair of articular cartilage defects using mesenchymal stem cells. Tissue Engineering 1995;1:345-53). Briefly, arthrotomies (using sterile technique) were performed on adult female rabbits that were at least 8 months old and the femoropatellar groove and medial condyle were exposed. The defects were drilled using a hand drill in the center of the femoropatellar groove and the medial condyle. The defects went through the articular cartilage and subchondral bone plate just to the trabecular bone. The drill bit was flat with a small triangular projection and the defects were drilled such that blood was just visible in the central dimple drilled by the projection but did not ooze from the defect. (Grande D A, Southerland S S, Manji R, Pate D W, Schwartz R E, Lucas P A. Repair of articular cartilage defects using mesenchymal stem cells. Tissue Engineering 1995;1:345-53). The defect recapitulates microfracture in that the drill does penetrate the subchondral bone, allowing access to the marrow. Thus empty defect is equivalent to microfracture and the PGA alone group is equivalent to placing matrix into a microfracture site.

There were two experimental treatment groups:
Group 1: Empty defect. The osteochondral defect was left empty as a control.
Group 2: PGA polymer alone group. The osteochondral defect was filled with sterilized PGA felt cultured in media alone for 2 weeks to serve as a vehicle control.

A total of 30 animals were used for this experiment. Half the animals, (15), were euthanized at 26 weeks post-op and the other half at 52 weeks post-op. Since both knees were utilized, there were 15 femoropatellar defects and 15 medial condyle defects for each of the 2 treatment groups per time point. Within each treatment group, 8 defects were used for histology and 7 were used for mechanical testing.

### Histology

After the animals were euthanized, those samples that were to be sent for histology had the distal femur removed (containing both defects) and fixed in 10% neutral buffered formalin+10% cetylpyridinium chloride. They were decalcified in Decal I (Fisher Scientific). At this point the femoropatellar groove was dissected from the medial condyles. Both the femoropatellar groove and medial condyle defects were bisected with a razor blade perpendicular to the long axis of the femur. The defects were then processed for paraffin histology with the center of the defect facing the microtome blade. Thus, when sectioning began, the sections were taken through the center of the defect. The remaining halves of the defects were stored in 70% ethanol. Sections of 7 μm thickness were obtained and stained with either Toluidine blue or Mallory-Heidenhain stain.

### Grading

Histological grading was done using the scale developed by O'Driscoll (O'Driscoll S W, Salter R B. The repair of major osteochondral defects in joint surfaces by neochondrogenesis with autogenous osteoperiosteal grafts stimulated by continuous passive motion. Clin Orthop Rel Res 1986; 208:131-140). At least 1 section per slide and 3 slides were graded per sample by two observers blinded as to the treatment group. The mean of all the scores was entered as the score for that sample. The O'Driscoll scale has a maximum value=25 for intact articular cartilage.

### Mechanical Testing

After euthanasia, animals whose defects were to be used for mechanical testing had their entire femurs dissected. All specimens were immediately stored at −20° C. until testing. Prior to testing they were thawed, keeping the distal femur covered with a phosphate buffered saline soaked cloth. The proximal end of the femur was rigidly fixed with polymethyl methacrylate using an alignment system to ensure specimen reproducibility.

All mechanical testing was done using an MTS 858 Mini Bionix (Eden Prairie, Minn.) bi-axial, servo-hydraulic materials testing apparatus. A 2 mm stainless steel probe was attached on the inferior actuator and a four jaw chuck was placed on the superior load cell. After the specimen was rigidly fixed in the testing apparatus and the 2 mm probe centered over the defect, the probe was advanced at a rate of 0.001 mm/s until a depth of 0.5 mm was obtained. Normal cartilage adjacent to the femoropatellar defect and the lateral condyle were also tested as an internal control to minimize variation between animals.

All data was downloaded into Excel format for data analysis. Force versus displacement graphs were generated for all tests and stiffness calculated from the linear portion of the graph as the probe compressed the defect/cartilage. The results were expressed as per cent stiffness of the defect to the stiffness of the control.

### Statistical Analysis

Histological grading was analyzed by Kruskal-Wallis and then pair-wise comparisons were by the Mann-Whitney U test, with a significance level of p<0.05. Mechanical data were analyzed by a one-way ANOVA and pairs of treatments by Tukey's post-hoc analysis. A significance level of p<0.05 was selected for the ANOVA and p <0.01 to the Tukey's. The lower significance level for the Tukey's was selected to avoid possible Type I errors due to the number of pair-wise analyses.

### Results

### 26 Week Post-Op

### Femoropatellar Groove

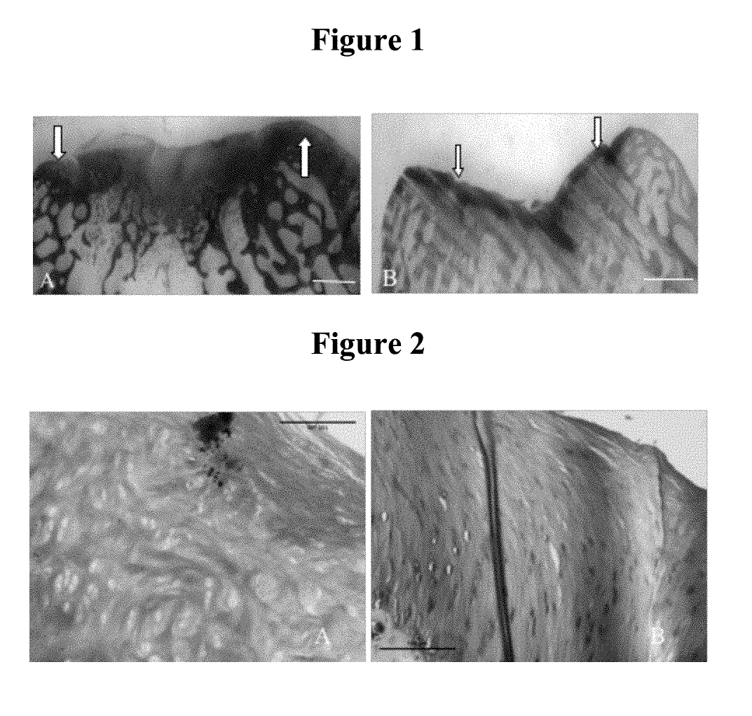

Empty defects at 26 weeks post-op were filled with either fibrocartilage or connective tissue that was not metachromatic with Toluidine blue staining, indicating a lack of proteoglycans (FIGS. 1A and 2A). Some defects had very little healing and the edges where the drill bit removed the original tissue were clearly visible (FIG. 1A). Defects in the polymer alone group at 26 weeks contained fibrocartilage and showed some regeneration of subchondral bone FIGS. 1B and 2B. During the operation the polymers were kept in 35 mm culture dishes containing PBS.

The histological scoring reflected the visual differences (Table 1) Empty defects had a mean score of 10.4 out of a possible score of 25 (complete regeneration); defects treated with PGA alone had a mean score of 8.8.

Mechanical testing of the femoropatellar groove revealed that the empty defects had mean compressive strength of only about 17% of the adjacent undamaged cartilage (Table 3). Defects treated with polymer alone had 31.7%.

### Medial Condyle

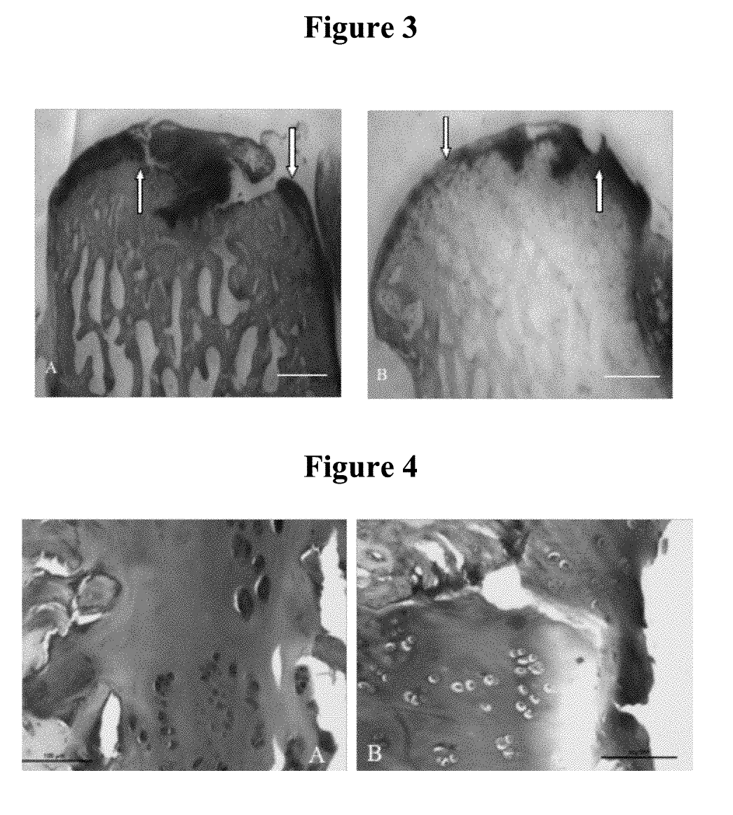

The histology of the medial condyle defects in the different treatment groups at 26 weeks post-op was very similar to that of the femoropatellar groove. Empty defects had predominantly connective tissue in the defect with some fibrocartilage present (FIG. 3A, 4A). The surface was irregular with tissue often extending beyond the adjacent cartilage surface into the joint space. There was poor integration of the repair tissue with the adjacent undamaged cartilage. Defects in the polymer alone group looked similar to the empty defects (FIG. 3B, 4B). Results are also shown in Table 1. Empty defects had a mean score of 9.5 out of a possible score of 25 (complete regeneration); defects treated with PGA alone had a mean score of 8.6.

Mechanical testing of the femoropatellar groove revealed that the empty defects had mean compressive strength of only about 17% of the adjacent undamaged cartilage (Table 3). Defects treated with polymer alone had 31.7%.

### 52 Weeks

### Femoropatellar Groove

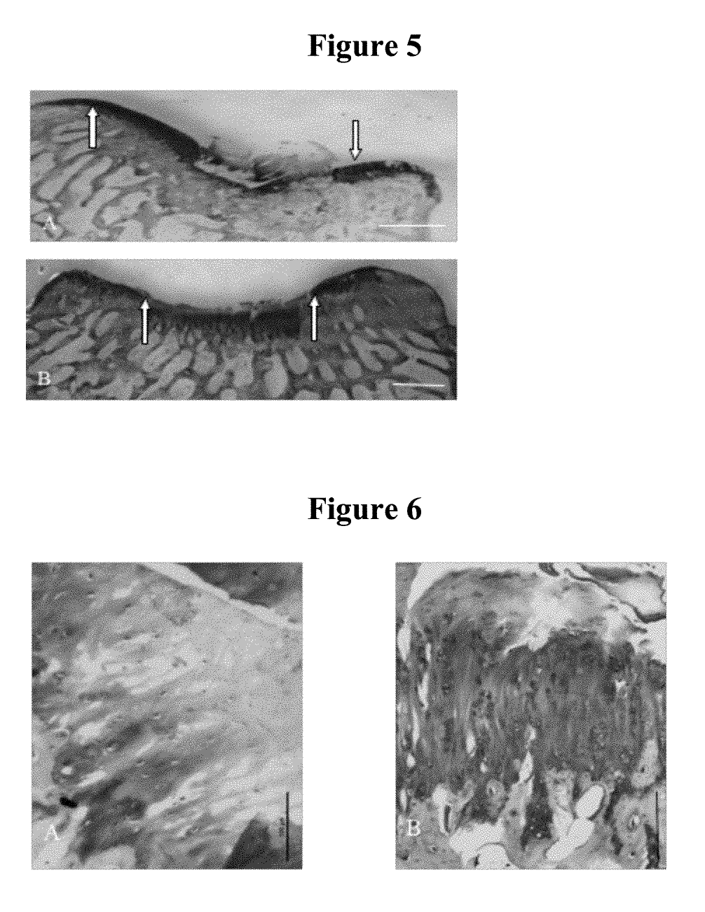

The histology at 52 weeks in the femoropatellar groove defects were essentially the same as observed at 26 weeks (see FIGS. 5 and 6 and Table 2). Both empty defects and defects treated with PGA alone contained loose connective tissue or some fibrocartilage. There had been no additional healing apparent in the intervening weeks.

The results for mechanical testing at 52 weeks in the femoropatellar groove were somewhat different from those of 26 weeks (See Tables 3 and 4). Empty defects had a mean mechanical strength of just 27.8% of adjacent cartilage. Defects treated with PGA alone had a value of 52.1%.

### Medial Condyle

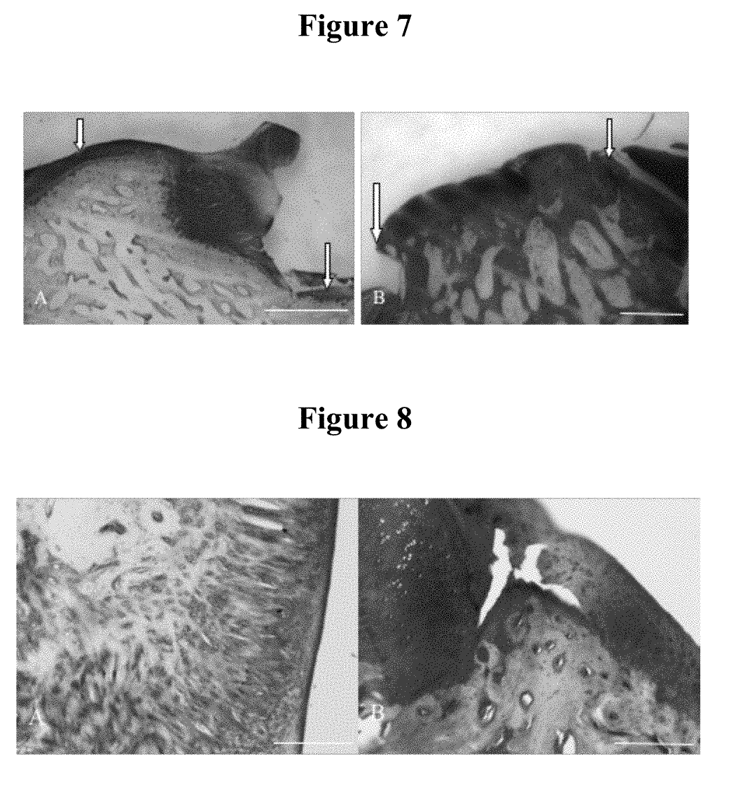

Results from these studies are shown in FIGS. 7 and 8 (histology) and in Tables 2 (histology) and 4 (mechanical strength). The histology at 52 weeks appears to be very similar to that observed at 26 weeks with respect to defects treated with PGA (a score of 8.6 out of a score of 24 at 26 weeks as opposed to a score of 9.4 at 52 weeks). The results for mechanical testing at 52 weeks was also similar to those reported for 26 weeks (46.6% v 37.7%).

TABLE 1
Histological Grading 26 Week Time Point

| | |
|---|---|
| Empty | PGA alone |

Femoropatellar Groove Treatment

| | | |
|---|---|---|
| Mean | 7.1 | 10.4 |
| SD | 1.5 | 1.9 |
| SEM | 0.5 | 0.7 |

Medial Condyle Treatment

| | | |
|---|---|---|
| Mean | 7.5 | 9.1 |
| SD | 2.4 | 3.5 |
| SEM | 0.8 | 0.4 |

TABLE 2
Histological Grading 52 Week Time Point

| | |
|---|---|
| Empty | PGA alone |

Femoropatellar Groove Treatment

| | | |
|---|---|---|
| Mean | 7.0 | 9.4 |
| SD | 2.4 | 2.5 |
| SEM | 2.5 | 0.9 |

Medial Condyle Treatment

| | | |
|---|---|---|
| Mean | 7.9 | 12.3 |
| SD | 1.6 | 3.5 |
| SEM | 0.6 | 1.2 |

TABLE 3
Mechanical Testing for 26 Week Time Point

| | |
|---|---|
| Empty | PGA alone |

Femoropatellar Groove Treatment

| | | |
|---|---|---|
| Mean | 17.6 | 31.7 |
| SD | 21.4 | 28.2 |
| SEM | 11.2 | 9.8 |

Medial Condyle Treatment

| | | |
|---|---|---|
| Mean | 26.6 | 37.7 |
| SD | 19.3 | 20.9 |
| SEM | 6.4 | 9.3 |

TABLE 4
Mechanical Testing for 52 Week Time Point

| | |
|---|---|
| Empty | PGA alone |

Femoropatellar Groove Treatment

| | | |
|---|---|---|
| Mean | 27.9 | 52.1 |
| SD | 6.4 | 20.3 |
| SEM | 2.3 | 7.7 |

Medial Condyle Treatment

| | | |
|---|---|---|
| Mean | 26.6 | 46.6 |
| SD | 6.9 | 10.2 |
| SEM | 2.4 | 3.9 |

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims.

Various references are cited throughout this specification, each of which is incorporated herein by reference in its entirety.

1. A method for growing new cartilage and optionally subchondral bone in a subject in need thereof comprising administering to a site where articular cartilage and optionally subchondral bone is needed an amount of a polymeric carrier suitable for proliferation and differentiation of autologous stem cells into articular cartilage or cartilage and subchondral bone, effective for growing said cartilage and said subchondral bone.

2. The method of claim 1 wherein the polymeric material is biodegradable.

3. The method of claim 1 wherein the polymeric material is formed of polymer fibers as a mesh or sponge.

4. The method according to claim 1, wherein the polymeric material is a biodegradable synthetic polymer.

5. The method of claim 3 wherein the polymeric material is a polyglycolic acid fibrous mesh.

6. The method of claim 1 wherein the said subject has a defect is in both cartilage and bone.

7. The method according to claim 1, wherein said method is combined with a microfracture technique to articular cartilage and/or subchondral bone in said subject.

8. The method according to claim 1, wherein said polymeric carrier is in the form of a polyglycolic acid felt disc.

9. The method according to claim 1, wherein said subject is afflicted with an osteochondral defect.

10. The method according to claim 1, where the polymer is incubated with a preparation comprising noncultured autologous stem cells prior to implantation at the osteochondral defect for a time sufficient for said autologous stem cells to attach to said polymer.

11. The method according to claim 1, wherein said preparation is derived from bone marrow.

12. The method according to claim 1, wherein said preparation is derived from adipose tissue.
